(19) United States
(12) Patent Application Publication
(10) Pub. No.: US 2013/0103032 A1
(43) Pub. Date: Apr. 25, 2013

(54) LAPAROSCOPIC ENDOMETRIOSIS ARTICULATING FULGURATOR

(71) Applicant: Richard B. Beaven, Covington, KY (US)

(72) Inventor: Richard B. Beaven, Covington, KY (US)

(21) Appl. No.: 13/277,271

(22) Filed: Oct. 20, 2011

(51) Int. Cl. A61B 18/18

(52) U.S. Cl. 606/48

(57) ABSTRACT

A surgical instrument includes a single articulating tip to apply energy to tissue within a body. The articulating tip travels in a semi-arc around an insulated hinge in such a way that the tip can articulate up or down less than 90 degrees from a central axis. In particular, the articulating range is between 70 degrees upward to about 20 degrees downward for a total range of movement of about 90 degrees. Other ranges of angles such as +60 degrees to −30 degrees are contemplated as well.

### BACKGROUND OF THE INVENTION

1. Field of the Invention

The present invention relates to medical instruments and, more particularly, to medical instruments having an articulating tip.

2. Description of Related Art

General surgical instruments are known that are inserted into a body, such as a human body, to manipulate in some way tissue within the body. These instruments have included scissors having two blades the pivot relative to one another to cut tissue and graspers that have two tips that close together in order to grasp tissue.

Endoscopic surgical instruments have been developed with varying degrees of complexity. In general endoscopic instruments are useful when they articulate up and down as well as left and right. However, to enable such complex articulation often requires complex mechanisms that are expensive and difficult to manufacture. Furthermore, endoscopic instruments are typically used by being inserted through an existing body orifice rather that a specific incision that places the instrument at a desired starting position.

There are also a number of ablation and fulgurator instruments that have been developed for specific purposes. Typically, these devices have two articulating tips that open or close but do not do so independent of one another. In other words, the top tip moves a certain distance away from a neutral position while at the same time the bottom tip moves the same distance but in an opposite direct from the neutral position. Such instruments are useful for many purposes and can be even be somewhat useful for unintended uses such a treating laparoscopic endometriosis. However, the two tips make such an instrument more prone to unintended contact with healthy tissue while being used.

In view of the surgical and medical instruments available today there still remains the need for a fulgurator having a single articulating tip that can be easily controlled and having an inexpensive and relatively simple articulating mechanism.

### BRIEF SUMMARY OF THE INVENTION

Embodiments of the present invention relate to a surgical instrument that includes a single articulating tip to apply energy to tissue within a body. The articulating tip travels in a semi-arc around a hinge in such a way that the tip can articulate up or down in an angle of about 90 degrees relative to a central axis of the instrument. In particular, the articulating range can be between about 70 degrees upward to about 20 degrees downward for a total range of movement of about 90 degrees. Other ranges of angles such as +60 degrees to −30 degrees are contemplated as well

It is understood that other embodiments of the present invention will become readily apparent to those skilled in the art from the following detailed description, wherein it is shown and described only various embodiments of the invention by way of illustration. As will be realized, the invention is capable of other and different embodiments and its several details are capable of modification in various other respects, all without departing from the spirit and scope of the present invention. Accordingly, the drawings and detailed description are to be regarded as illustrative in nature and not as restrictive.

### BRIEF DESCRIPTION OF DRAWINGS

FIG. 1 illustrates a block diagram of aspects of the present invention.

FIG. 2 illustrates a side view of a surgical instrument in accordance with the principles of the present invention.

FIG. 3 illustrates a side view of the instrument of FIG. 1 in which the trigger is moved to articulate the tip downwards.

FIG. 4 illustrates a side view of the instrument of FIG. 1 in which the trigger is moved to articulate the tip upwards.

FIG. 5A-FIG. 5D illustrate details of an articulating tip in accordance with the principles of the present invention.

FIG. 6 illustrates a flowchart of an exemplary method of using the articulating tip instrument in accordance with the principles of the present invention.

### DETAILED DESCRIPTION OF INVENTION

The detailed description set forth below in connection with the appended drawings is intended as a description of various embodiments of the invention and is not intended to represent the only embodiments in which the invention may be practiced. The detailed description includes specific details for the purpose of providing a thorough understanding of the invention. However, it will be apparent to those skilled in the art that the invention may be practiced without these specific details. In some instances, well known structures and components are shown in block diagram form in order to avoid obscuring the concepts of the invention.

In the description provided herein, the phrase “upward” and “downward” are used to aid the reader in understanding how portions of the instrument move relative to one another. These terms, however, depend on the orientation of the instrument relative to an arbitrary set of three-dimensional axes. Thus, if the instrument were rotated along one or more of these axes, then the relative movement of the instrument portions to one another remain the same but “upwards” is no longer the same relative to the arbitrary axes. Accordingly, the terms “upwards” and “downwards” are not meant to limit the operating parameters of the present invention but are merely provided so that, with the aid of the drawings and their fixed perspective, a reader can more easily understand how portions of the articulating instrument move relative to one another. Thus, the terms “upwards” and “downwards” are merely convenient terms to describe how, within its plane of rotation, the articulating tip can deflect in one direction from a neutral position and can also deflect in an opposite direction. Similarly, the instrument will often times be referred to as a fulgurator, however, one of ordinary skill will recognize that this term does not encompass all the functionality that can be performed such as sealing tissue, coagulating tissue, dissecting tissue, freezing tissue, etc. Thus, the term “fulgurator” also is not intended to limit embodiments of the present invention to only destruction of living tissue by electric sparks generated by a high-frequency current.

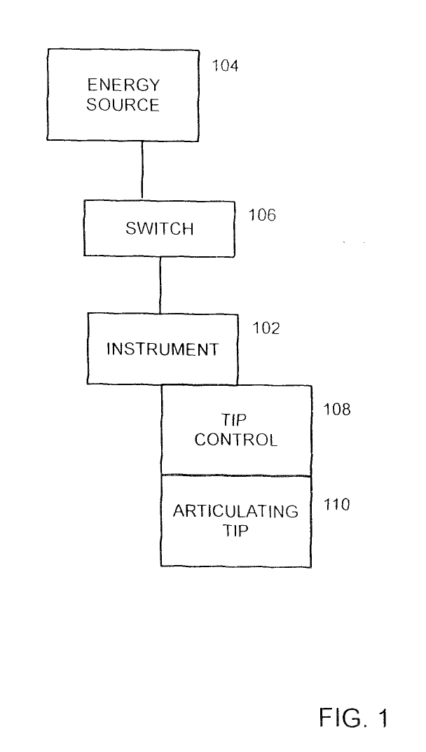

FIG. 1 illustrates a block diagram of aspects of the present invention. The instrument 102 is shaped and sized for insertion into an incision made in the human body, typically with the help of a trocar. Not shown in FIG. 1 are one or more cameras that may also be used in conjunctions with the instrument 102 to aid the user in positioning the instrument 102 as desired. The purpose of inserting the instrument 102 into an incision is so that part of the instrument 102 can be positioned within an internal cavity of the body such as the abdominal cavity. Once in the cavity, the instrument 102 can be used to manipulate tissue such as, for example, endometrial cells outside the uterus.

A portion of the instrument 102 is located outside of the body cavity, such as in a surgeon's hand, and this portion includes a tip control mechanism 108 that allows the surgeon to move the tip as desired. Thus, gross-level positioning of the instrument 102 can position it close to a desired location while fine-level tip positioning can be used to place the articulating tip 110 at a precise location.

Once the articulating tip is in its desired location, then a switch 106 can be triggered that will cause energy from an energy source 104 to be communicated with the articulating tip 110 so that the energy is delivered to the body tissue where the articulating tip 110 is located. One typical type of energy that is used for fulguration is high frequency electrical current (e.g., from about 9 kHz to 1000 GHz). This electrical current can be delivered using a bi-polar tip or a unipolar tip. As is known, the unipolar tip (and energy source) completes the electrical circuit through another portion of the patient's body, while a bi-polar tip (and energy source) provides both the positive and ground paths for the electrical current to travel. In addition to electrical signals, other ways of cutting, coagulating, ablating and fulgurating tissue are known such as through a vibrating ceramic that uses friction to affect the tissue that it touches. There are also known frequencies and waveforms that have different effects such that a desired duration and type of energy can be delivered by controlling the energy source 104 accordingly. Embodiments of the present invention do not require any particular energy source 104 or switch 106 to operate according to its inventive principles. Thus, one of ordinary skill will recognize that the instrument 102 described herein can be utilized with a variety of different energy source without departing from the scope of the present invention. For example, an instrument having an articulating tip operating in accordance with the principles of the present invention may be used to perform cryotherapy as well wherein the tip delivers cryogenic freezing temperatures to tissue within a body cavity.

Because the instrument 102 and articulating tip 110 are intended to be inserted within the human body, they are constructed of materials selected to withstand that environment and to be safely used within that environment. While various recipes of stainless steel are useful for some portions of the instrument 102 and tip 110, insulative plastic and polymer materials may also be used as appropriate and further described herein. It is envisioned that a wide range of materials can be selected based on their known properties such that the portion of the instrument 102 within the body is of an inert material that has minimal unintended effect on bodily tissue and that the tip can deliver energy at a desired location but, also not impact any unintended areas.

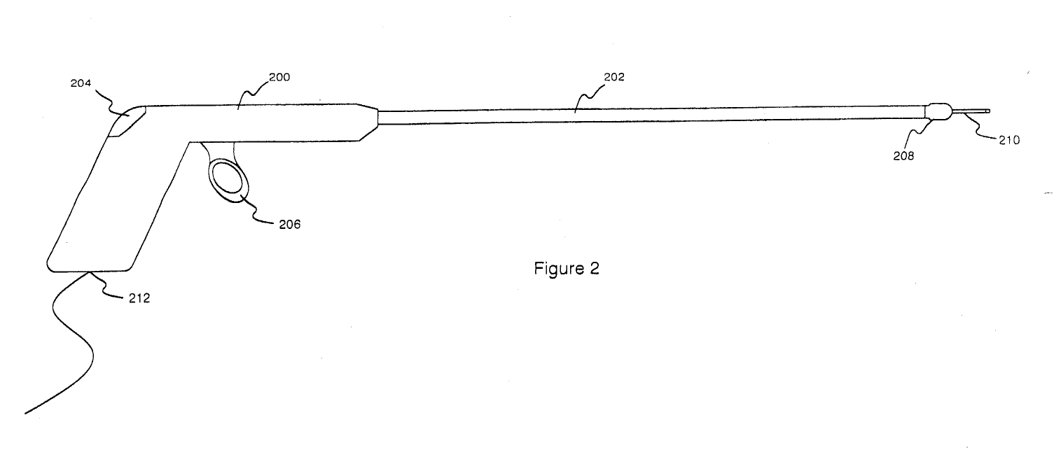

FIG. 2 illustrates a side view of a surgical instrument in accordance with the principles of the present invention. There is a handle portion 200 that includes a trigger 206 that controls movement of the articulating tip 210. The handle portion also includes a port 212 that accepts a signal from an energy source (not shown) and a switch 204 that activates or deactivates delivery of the energy source signal to the tip 210. The switch 204 is shown on the handle portion 200 in FIG. 2 but the switch 204 can also be a floor-mounted switch or mounted to some other surface as well.

The handle portion 200 is connected to a proximal end of a shaft 202 and at the distal end of the shaft 202 is an articulating mechanism 208, such as for example, a hinge and a tip 210. The trigger 206 is used to control movement of the articulating mechanism 208 such that the tip 210 can move upwards or downwards. In a particular embodiment, the tip 210 is rigid such that it does not bend but, instead rotates as a rigid shaft about a hinge 208.

The trigger 206 and articulating mechanism 208 can be connected in a variety of different ways to effect mechanical movement of the tip 210 in a desired manner. For example, electromagnetic motors could be used at the trigger 206, the articulating mechanism 208, or both to effect movement of the tip 210. A variety of mechanical movements such a screw gears, planetary gears and other interlocking geared shafts could also be used to affect the desired movement of the tip 210. While such complex approaches may be useful in certain environments, a simple mechanical linkage from the trigger 206 to the articulating mechanism 208 is a beneficial approach that simplifies the design of the instrument shown in FIG. 2. Similarly, the articulating mechanism 208 can be any of a wide variety of designs such that the tip 210 can pivot relative to the shaft 202. However, a simple hinge having a fixed portion and a rotating portion either encircling the fixed portion or encompassed within the fixed portion also simplifies the design of the instrument shown in FIG. 2.

Although shown as a trigger 206, this mechanism that the surgeon manipulates to control the degree of articulation of the tip 210 can be constructed in other ways as well. For example, a thumbwheel can be used that the surgeon rotates back a forth. Also, a sliding switch can be used that slides back and forth to effect motion of the tip 210. In some instances, a trackball or joystick mechanism could also be used wherein the movement of the manipulated mechanism is transformed into appropriate movement of the articulating tip 210 such that the tip is positioned at a desired angle and location.

As for size, the tip 210 can be selected for different purposes and can range from about 2 cm to 4 cm in length. The shaft 202 can vary in size as well but a length of about 40 cm and a diameter which can be of different sizes will allow a variety of uses. Thus, a wide variety of shaft diameters is beneficial. The shaft of the instrument is typically inserted through a plastic tube placed through an incision in the skin. These tubes, known as trocars, come in sizes ranging from 5 to 15 mm. Thus, the diameter of the shaft 202 can vary along that same range of sizes as well. The handle portion 200 can be any of a variety of different designs that allow it to be held by a single hand in a steady manner that provides easy and intuitive access to the trigger 206.

The trigger 206 may be configured such that constant pressure by a finger is needed to hold the trigger 206 in a desired position. Thus, a spring can be attached to the trigger 206 biased in such a way as to return the trigger 206 to a neutral position so that pressure by a finger is required to hold the trigger 206 in a forwards or backwards position and thus to hold the articulating tip 110 in a desired angle. Alternatively, a ratcheting mechanism can be employed so that movement forward of the trigger ratchets the mechanical linkage in one direction and movement backwards of the trigger 206 ratchets the mechanical linkage in an opposite direction. In this way, the articulating tip is positioned at a desired angle and remains there even if the trigger 206 is released.

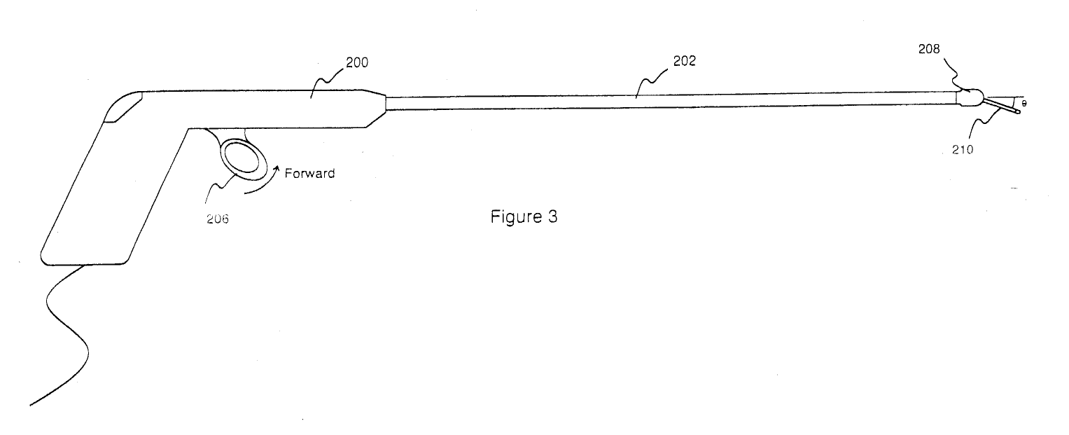

FIG. 3 illustrates a side view of the instrument of FIG. 1 in which the trigger is moved to articulate the tip downwards. In FIG. 2, the trigger 206 was in a position that placed the tip 210 in a neutral position. In this position the tip 210 has its major axis substantially aligned with the major axis of the shaft 202. In FIG. 3, however, the trigger 206 is moved so that the tip 210 articulates downwards an angle θ. The articulating mechanism 208 and the tip 110 are constructed such that the maximum angle downwards that the tip 210 can move is about 20 degrees. This limited movement can be accomplished using a variety of methods. For example, the length, and thus allowed movements, of the mechanical linkages between the trigger 206 and the tip 210 can be selected so that maximum mechanical travel of the trigger 206 forward would result in tip articulation downward of only 20 degrees. Thus moving the trigger from a neutral position to its maximum forward position would articulate the tip 210 downwards from zero degrees to 20 degrees. Alternatively, or in addition to, mechanical stops can be present which stop the movement of the articulating tip 210 at a desired maximum downward angle.

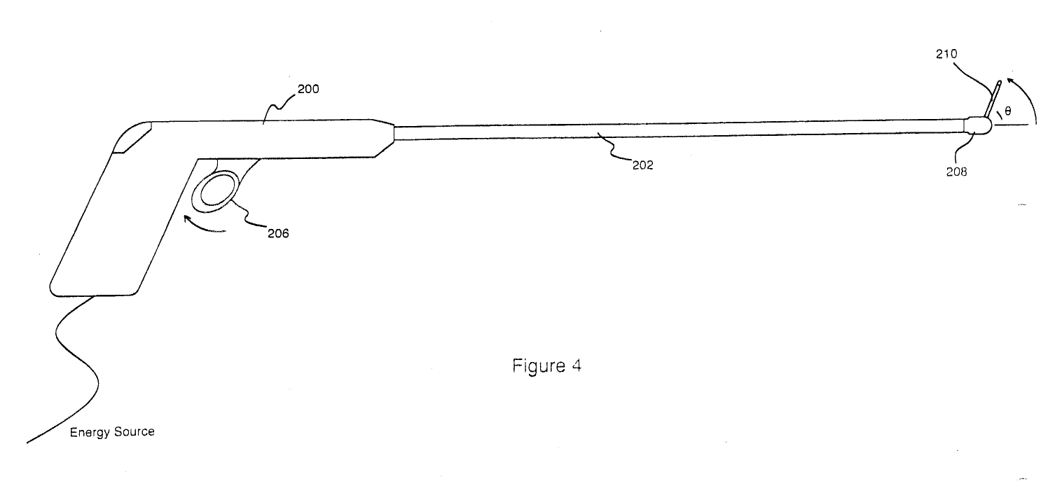

FIG. 4 illustrates a side view of the instrument of FIG. 1 in which the trigger is moved to articulate the tip upwards. In FIG. 3, the trigger 206 was in a position that placed the tip 210 in a downward position. In this position the tip 210 has its major axis tilted, or deflected, downward with respect to the major axis of the shaft 202. In FIG. 4, however, the trigger 206 is moved so that the tip 210 articulates upwards an angle θ. In this position the tip 210 has its major axis tilted, or deflected, upward with respect to the major axis of the shaft 202. The articulating mechanism 208 and the tip 110 are constructed such that the maximum angle upwards that the tip 210 can deflect is about 70 degrees. This limited movement can be accomplished using a variety of methods. For example, the length, and thus allowed movements, of the mechanical linkages between the trigger 206 and the tip 210 can be selected so that maximum mechanical travel of the trigger 206 backward would result in tip articulation upward of only 70 degrees. Thus moving the trigger from a neutral position to its maximum backward position would articulate the tip 210 upwards from zero degrees to 70 degrees. Alternatively, or in addition to, mechanical stops can be present which stop the movement of the articulating tip 210 at a desired maximum upwards angle.

Thus, the instrument of FIG. 2-4 includes only one articulating tip that has a limited range of articulation angles. In particular the articulating tip can rotate downwards to about −20 degrees and upwards to about 70 degrees. These specific angles provide what is believed to be a beneficial range of articulation; however other ranges are contemplated as well. Because a surgeon can rotate the instrument and, thereby “change” the plane of reference as needed to reach any particular tissue within the body cavity, the total beneficial range of articulation for the single tip instrument to be achieved is about 90 degrees. In the specific embodiment described above, the range of 90 degrees was accomplished with a possible 70 degree movement in one direction from a neutral position and a possible 20 degree movement in the opposite direction. However, a range of articulation from about +60 degrees to −30 degrees will also provide the desired total range of 90 degrees. While +45 degrees to −45 degrees would also provide the desired total range of articulation, it is beneficial to have articulation in one direction to be closer to 90 degrees as this allows the tip to more easily reach tissue that are oriented somewhat orthogonally to the major axis of the shaft 202.

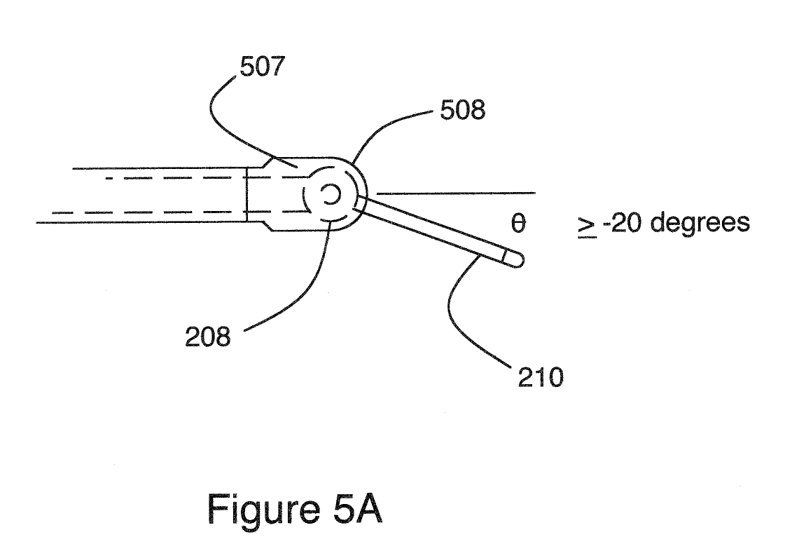

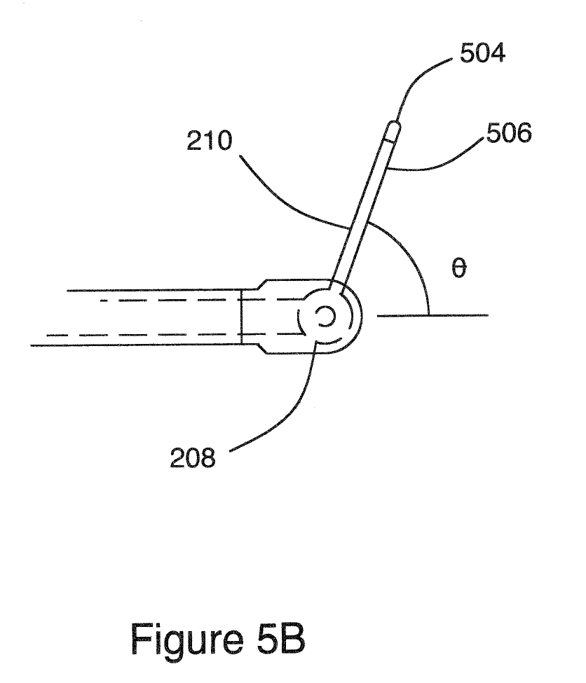

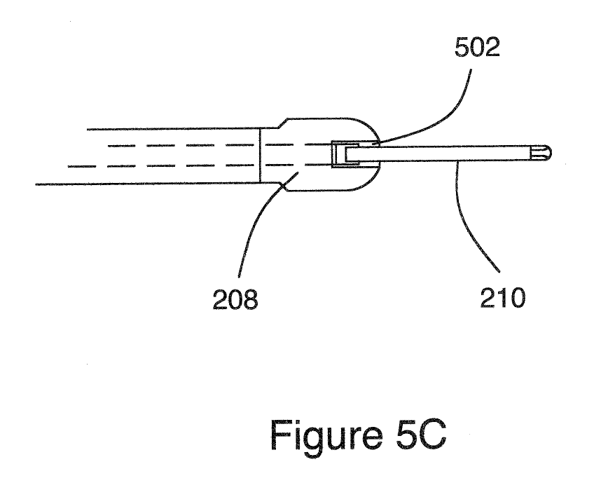

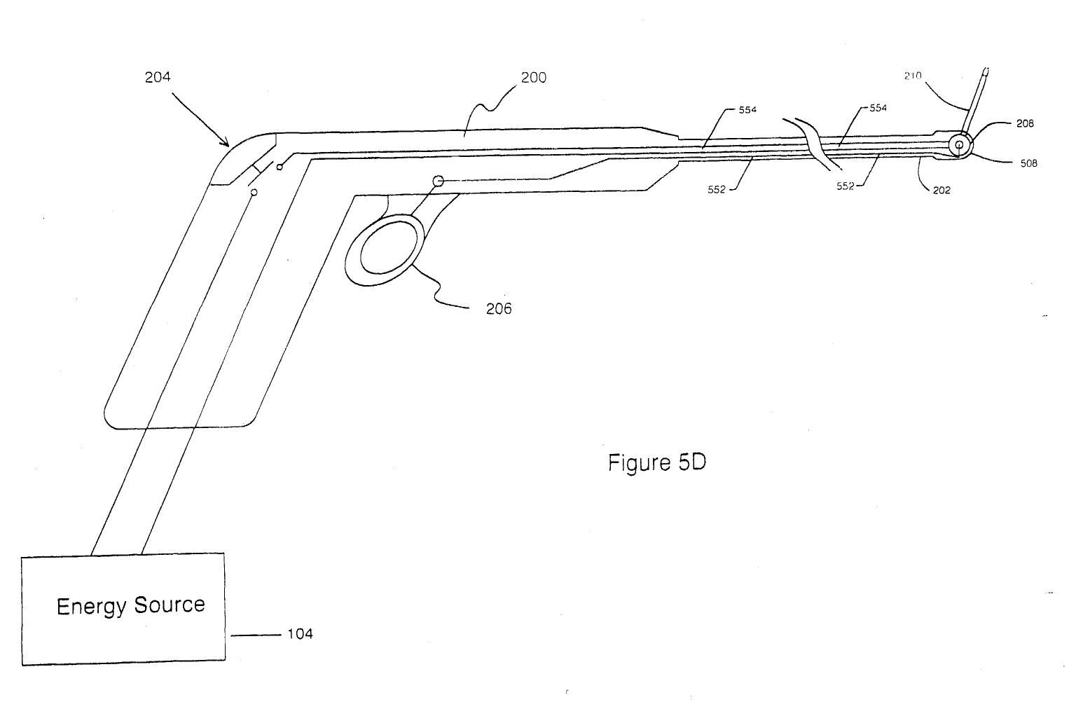

FIG. 5A-FIG. 5D illustrate details of an articulating tip in accordance with the principles of the present invention. In FIG. 5A-5C the articulating mechanism 208 is shown to be enclosed in an insulating cap 508. In particular, the tip 210 includes a shaft 506 and a distal end 504 that are conductive for the energy being delivered from the energy source 104 (See FIG. 1). Thus, there is a portion of the articulating mechanism 208 around the region 507 that would be exposed if not for the insulating cap 508. Without the insulating cap 508, there would be a greater risk that unintended tissue could be contacted by portions of the tip 210 that deliver energy to the body tissue. Thus, a substantial portion of the articulating mechanism may be covered by the electrically insulating cap 508. By “substantial portion” it is meant that as much of the articulating mechanism can be covered without interfering with the operation of the articulating mechanism and the articulating tip. The top view from FIG. 5C shows an opening, or slot, 502 in the cap 508 that allows the tip 210 to move but also prevents the majority of the articulating mechanism 208 from being exposed. As mentioned briefly above, the slot 502 can be sized so that it provides a lower hard mechanical stop and an upper hard mechanical stop that prevent the tip 210 from articulating outside of the desired range of articulation.

FIG. 5D shows a cutaway view inside the instrument of FIG. 2. From this view it can be seen that the energy source 104 is connected through a conductive path 554 and the switch 204 to the articulating tip 210. Additionally, a trigger 206 is also coupled through mechanical linkages 552 to the articulating mechanism 208. An insulating cap 508 helps minimize the amount of the articulating tip 210 and articulating mechanism 208 that are exposed. In this way, unintended damage to tissue may be reduced. The end of the tip 210 may be either a unipolar end or a bi-polar end depending on the energy source 104 selected. The difference being that a bi-polar tip includes both the positive and negative electrodes in close proximity to one another. Other types of tips can be used as well if other energy sources are selected such as, for example, tips used with harmonic energy sources.

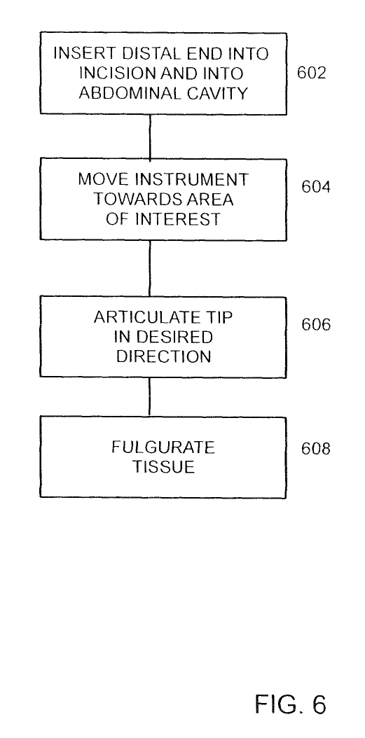

FIG. 6 illustrates a flowchart of an exemplary method of using the articulating tip instrument in accordance with the principles of the present invention. Because of the position of the patient's body during endometriosis fulguration as well as the structure of the abdominal cavity, there are regions that are more easily seen and reached with a non-articulating fulgurator. For some regions, however, a direct or straight approach is difficult and damage to surrounding tissue can occur or complete fulguration may be difficult. Thus, an articulating tip fulgurator as described above is useful in many instances. However, using dual-tipped instruments as a makeshift fulgurator is difficult in many instances because the risk of unintentionally damaging nearby tissue increases. Accordingly, a method is described with reference to FIG. 6 that uses the single articulating tip device described above to perform fulguration of tissue such as endometrial cells within the abdominal cavity. One of ordinary skill will recognize that the method and instrument can be used for other tissue-related surgical procedures as well without departing from the scope of the present invention.

Step 602 assumes that an incision has previously been made through which to insert the distal tip of a surgical instrument in accordance with the principles of the present invention. Also, it is assumed that there are one or more cameras present that image the area within the body where the distal tip may be moved around in order to fulgurate desired tissue. Although such a camera is assumed to be separate from the device illustrated in FIGS. 2-5D, the camera could be located near the articulating tip 210 such that the image signal is acquired by a camera connected to the surgical instrument with the image signal being sent through a cable within the shaft 202 or possibly by a wireless transmitter to a viewing screen outside the body. In step 602, the distal, articulating end of the surgical instrument is inserted into the incision and thus into the abdominal cavity of a person (or animal).

In step 604, the instrument is moved in such a way that the distal end is positioned near tissue that is to be fulgurated. Once in the general desired location, the articulating tip at the distal end of the instrument can be articulated, in step 606, to the desired angle such that a significant portion of the tip surface is in contact with the tissue to be fulgurated. Depending on the orientation of the person and the tissue within the abdominal cavity, the tip is articulated to an angle within −20 degrees to +70 degrees from a neutral position that best locates the tip to perform fulguration without unintentionally damaging nearby tissue. Once in place, the surgeon, in step 608, delivers energy through the instrument to the tissue such that the tissue is fulgurated.

The previous description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein, but are to be accorded the full scope consistent with each claim's language, wherein reference to an element in the singular is not intended to mean “one and only one” unless specifically so stated, but rather “one or more.” All structural and functional equivalents to the elements of the various embodiments described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. §112, sixth paragraph, unless the element is expressly recited using the phrase “means for” or, in the case of a method claim, the element is recited using the phrase “step for.”

What is claimed is:

1. A surgical instrument comprising:
a handle portion including a trigger;
a shaft having a proximal end, a distal end, and a major axis, the proximal end coupled with the handle;
an articulating mechanism coupled with the distal end of the shaft and in mechanical communication with the trigger such that movement of the trigger controls an angle of the articulating mechanism about an axis of rotation, the axis of rotation substantially orthogonal to the major axis of the shaft;
only one tip coupled with the articulating mechanism, the tip having a tip shaft, a proximal tip end coupled with the articulating mechanism, and a distal tip end extending away from the articulating mechanism such that rotation of the articulating mechanism causes the tip shaft to deflect an angle away from the major axis of the shaft; wherein the deflection angle is restricted within a range of about 90 degrees having a first maximum deflection angle in a first direction away from the major axis of the shaft and a second maximum deflection angle in an opposite direction from the first direction; and
an energy source in electrical communication with the distal tip end configured so that energy from the energy source is communicated to the distal tip end.

2. The instrument of claim 1, further comprising:
a switch between the distal tip end and the energy source to determine when energy is communicated to the distal tip end and when energy is interrupted.

3. The instrument of claim 2, wherein the handle portion includes the switch.

4. The instrument of claim 1, wherein the only one tip is a unipolar tip.

5. The instrument of claim 1, wherein the only one tip is a bi-polar tip.

6. The instrument of claim 1, further comprising:
an enclosure located at the distal end of the shaft and configured to cover substantially all of the articulating mechanism, wherein the enclosure is constructed from an electrically insulative material.

7. The instrument of claim 6, wherein the enclosure further comprises:
a slot through which the tip shaft passes as it deflects away from the major axis of the shaft.

8. The instrument of claim 7, wherein the slot is sized such that it has a top surface that stops deflection of the tip shaft in the first direction and a bottom surface that stops deflection of the tip shaft in the opposite direction.

9. The instrument of claim 1, wherein the trigger has a neutral position such that when located in the neutral position a major axis of the tip shaft is substantially aligned with the major axis of the shaft.

10. The instrument of claim 1, wherein the trigger is hinged so as to rotate forward and backward around an axis of rotation substantially orthogonal to the major axis of the shaft.

11. The instrument of claim 10 wherein:
when the trigger is rotated a maximum amount forwards, the tip shaft is deflected downwards about 20 degrees from the major axis of the shaft; and
when the trigger is rotated a maximum amount backwards, the tip shaft is deflected upwards about 70 degrees from the major axis of the shaft.

12. The instrument of claim 1, further comprising:
a mechanical link between the trigger and the articulating mechanism configured so that movement of the trigger is translated into deflection of the tip shaft.

13. The instrument of claim 1, wherein the articulating mechanism is a hinge.

14. A method of fulgurating tissue comprising:
positioning a surgical instrument within a cavity of a body, wherein the surgical instrument comprises:
a handle portion including a trigger;
a shaft having a proximal end, a distal end, and a major axis, the proximal end coupled with the handle;
an articulating mechanism coupled with the distal end of the shaft and in mechanical communication with the trigger such that movement of the trigger controls an angle of the articulating mechanism about an axis of rotation, the axis of rotation substantially orthogonal to the major axis of the shaft;
only one tip coupled with the articulating mechanism, the tip having a tip shaft, a proximal tip end coupled with the articulating mechanism, and a distal tip end extending away from the articulating mechanism such that rotation of the articulating mechanism causes the tip shaft to deflect an angle away from the major axis of the shaft; wherein the deflection angle is restricted within a range of about 90 degrees having a first maximum deflection angle in a first direction away from the major axis of the shaft and a second maximum deflection angle in an opposite direction from the first direction;
operating the trigger to deflect the tip shaft a first angle;
positioning the distal tip end to be in contact with tissue within the cavity; and
fulgurating the tissue by applying an energy source to the distal tip end.

15. The method of claim 14, wherein the only one tip is a unipolar tip.

16. The method of claim 14, wherein the only one tip is a bi-polar tip.

17. The method of claim 14, further comprising:
providing an enclosure that covers a substantial portion of the articulating mechanism with an electrically insulating material.

18. The method of claim 17, wherein the enclosure further comprises:
a slot through which the tip shaft passes as it deflects away from the major axis of the shaft.

19. The method of claim 18, wherein the slot is sized such that it has a top surface that stops deflection of the tip shaft in the first direction and a bottom surface that stops deflection of the tip shaft in the opposite direction.

20. The instrument of claim 1, wherein the first maximum deflection angle is about 70 degrees and the second maximum deflection angle is about 20 degrees.

21. The instrument of claim 1, wherein the first maximum deflection angle is about 60 degrees and the second maximum deflection angle is about 30 degrees.
